(19) United States
(12) Patent Application Publication
(10) Pub. No.: US 2011/0250642 A1
(43) Pub. Date: Oct. 13, 2011

(54) Method for Displaying Antibodies

(75) Inventors: Henry Ji, San Diego, CA (US); Heyue Zhou, San Diego, CA (US); Yanliang Zhang, San Diego, CA (US); Charles Rodi, Del Mar, CA (US)

(73) Assignee: Sorrento Therapeutics

(21) Appl. No.: 13/085,381

(22) Filed: Apr. 12, 2011

Related U.S. Application Data
(60) Provisional application No. 61/345,895, filed on May 18, 2010.
(60) Provisional application No. 61/323,218, filed on Apr. 12, 2010.

(51) Int. Cl. C12P 21/00

(52) U.S. Cl. 435/69.6

(57) ABSTRACT

There is disclosed vector designs, constructs and approaches to construct antibody libraries that display antibodies, such as full-length immunoglobulins, single chain antibody (SCA) scFv, or Fab on the host cell surface. There is also disclosed screening approaches to isolate desired antibody binders from above mentioned antibody libraries for selective antibody targets.

### CROSS REFERENCE TO RELATED APPLICATIONS

The present disclosure claims priority from U.S. Provisional Patent Application 61/323,218 “Novel Methods of Displaying Antibodies” filed 12 Apr. 2010 and U.S. Provisional Patent Application 61/345,895 entitled “Novel Methods of Displaying Antibodies” filed on 18 May 2010. The disclosures of both provisional patent applications are incorporated by reference herein.

### TECHNICAL FIELD

The present disclosure provides vector designs, constructs and approaches to construct antibody libraries that display antibodies, such as full-length immunoglobulins, single chain antibody (SCA) scFv, or Fab on the host cell surface. The present disclosure also provides screening approaches to isolate desired antibody binders from above mentioned antibody libraries for selective antibody targets.

### BACKGROUND

Antibodies are valuable, both as therapeutic agents and as general reagents in a variety of molecular biological processes. Methods of producing polyclonal and monoclonal antibodies are available, as are many antibodies. A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975.

Naturally occurring antibodies, or immunoglobulins (Igs), comprise a basic four polypeptide chain structure comprising two identical heavy (H) chains and two identical light (L) chains which are stabilized and cross-linked by intra-chain and inter-chain disulphide bonds. Different antibody classes comprise variants of this four-chain structure. Each heavy chain comprises a variable domain at its N-terminal followed by several constant domains. Each light chain has a variable domain at its N-terminal and one constant domain at its C-terminal. Because the largest amount of sequence variation is concentrated in the N-terminal domains of the light and heavy chains; each of these domains is termed a variable (V) domain (or “V region”). The constant domains make up the constant region, which comprises the remainder of the molecule and exhibits relatively little sequence variation. Heavy chains are comprised of five major types, depending on the antibody class, and consist of about 450-600 amino acid residues. Light chains are of two major types and have about 230 amino acid residues. Both heavy and light chains are folded into domains, comprising globular polypeptide regions.

Antibodies typically comprise two large heavy chains and two small light chains. There are five types of mammalian Ig heavy chain. They are denoted by the Greek letters: α, δ, ε, γ, and μ. The type of heavy chain present defines the class of antibody—IgA, IgD, IgE, IgG, and IgM. Each heavy chain comprises a constant region and a variable region. There are two types of light chains—lambda (λ) and kappa (κ). Each light chain comprises a constant region and a variable region. The constant region is identical in all antibodies of the same isotype. The variable region differs in antibodies produced by different B cells, but is identical for all antibodies produced by a single B cell.

The portion of the antibody that binds to an antigen (the antigen binding site) is contained in the Fab (fragment, antigen binding) region. The Fab region is composed of (a) a heavy chain constant and variable domain, and (b) a light chain constant and variable domain. The variable domain is referred to as the FV region. The variable domain on the light chain is abbreviated as VL. The variable domain on the heavy chain is abbreviated as VH.

The portion of the antibody that modulates immune cell activity is called the Fc (Fragment, crystallizable) region. The Fc region is composed of the constant regions of two heavy chains.

In the antibody, the variable domain of the light chain is aligned with the variable domain of the heavy chain; the constant domain of the light chain is aligned with the first constant domain of heavy chain. The variable domains of each pair of light and heavy chains form the antigen binding site for binding the antibody to an epitope of the antigen. The constant domains in the light and heavy chains are not directly involved in antigen binding. Each heavy or light chain variable domain comprises four relatively conserved framework (FR) regions (or framework segments) which are separated and connected by three hypervariable or complementarity determining regions (CDRs), which are believed to contact the target antigen of the antibody and to be principally responsible for binding of the antibody to the antigen.

The framework regions and CDRs have been defined. (Kabat et al., SEQUENCES OF PROTEINS OF IMMUNOLOGICAL INTEREST, U.S. Dept. Health and Human Services, National Institutes of Health, USA (5th ed. 1991); and Wu et al., J. Exo. Med. 132:211-250 (1970), each of which is incorporated herein by reference in its entirety for all purposes. For additional discussion of the structure of variable domains, see Poljak et al., Proc. Natl. Acad. Sci. USA, 70:3305-3310, 1973; Segal et al., Proc. Natl. Acad. Sci. USA, 71:4298-4302, 1974; and Marquart et al., J. Mol. Biol., 141, 369-391, 1980, each of which is incorporated herein by reference in its entirety for all purposes. The sequences of the framework regions of different light or heavy chains are relatively conserved within a species. The combined heavy and light chain framework regions of an antibody serve to position and align the CDRs for proper binding to the antigen.

The amino acids of the CDRs of the variable domains were initially defined based on sequence variability, to consist of amino acid residues 31-35 (H1), 50-65 (H2), and 95-102 (H3) in the human heavy chain variable domain (VH) and amino acid residues 24-34 (L1), 50-56 (L2), and 89-97 (L3) in the human light chain variable domain (VL), using Kabat's numbering system for amino acid residues of an antibody. (Kabat et al., SEQUENCES OF PROTEINS OF IMMUNOLOGICAL INTEREST, U.S. Dept. Health and Human Services, NIH, USA (5th ed. 1991). Chothia and Lesk, J. Mol. Biol. 196:901-917, 1987) presented another definition of the CDRs based on residues that included in the three-dimensional structural loops of the variable domain regions, which were found to be important in antigen binding activity. Chothia and Lesk defined the CDRs as consisting of amino acid residues 26-32 (H1), 53-55 (H2), and 96-101 (H3) in the human heavy chain variable domain (VH), and amino acid residues 26-32 (L1), 50-52 (L2), and 91-96 (L3) in the human light chain variable domain (VL). Combining the CDR definitions of Kabat and Chothia and Lesk, the CDRs consist of amino acid residues 26-35 (H1), 50-65 (H2), and 95-102 (H3) in human VH and amino acid residues 24-34 (L1), 50-56 (L2), and 89-97 (L3) in human VL, based on Kabat's numbering system.

V genes encode the approximately N-terminal 95 amino acids of the V domains. The number of V genes at each locus varies between loci and species, but may include up to about several hundred V genes.

Antibody heavy chain V domains include V genes, D (diversity) genes, and J (joining) genes. The large diversity in antibody variable domains results from, in part, recombination between V, D, and J gene segments. To produce a gene encoding a heavy chain variable domain, any one of the heavy chain variable domain genes is recombined with any one of a small number of D and J genes to produce a VDJ gene. The recombination process of a light chain variable domain is similar, except that a V gene is recombined directly with a J gene, since light chain variable domains have no D gene segments.

In terms of antibody libraries, generally, such libraries have begun with initial screening of murine libraries to find a murine antibody having the desired binding affinities within its variable domain regions of its heavy and light chains, otherwise called the Fab region. However, such murine antibodies would not be useful for human therapeutics as administration of a murine-derived antibody would cause an immune or rejection response by the host. U.S. Pat. No. 5,869,619 discloses a possible procedure for reducing the immunogenicity of antibody variable domains while preserving their ligand binding properties in which characteristically human residues are substituted for murine variable domain residues that are determined or predicted (i) to play no significant chemical role in the interaction with antigen, and (ii) to be positioned with side chains projecting into the solvent, Thus, exterior residues remote from the antigen binding site are humanized, while interior residues, antigen binding residues, and residues forming the interface between variable domains remain murine. One disadvantage of this approach is that rather extensive experimental data is required to determine whether a residue plays no significant chemical role in antigen binding or will be positioned in the solvent in a particular three dimensional antibody structure.

In U.S. Pat. No. 5,225,539 (Winter I) contiguous tracts of murine variable domain peptide sequence considered conserved are replaced with the corresponding tracts from a human antibody. In this more general approach, variable domain residues are humanized except for the non-conserved regions implicated in antigen binding. To determine appropriate contiguous tracks for replacement, a classification of antibody variable domain sequences was used that had been developed previously by Wu and Kabat (1970).

The Winter I humanization method used the Kabat classification results in a chimeric antibody comprising CDRs from one antibody and framework regions from another antibody that differs in species origin, specificity, subclass, or other characteristics. However, no particular sequences or properties were ascribed to the framework regions, Winter I taught that any set of frameworks could be combined with any set of CDRs. Framework sequences have since been recognized as being important for conferring the three dimensional structure of an antibody variable region necessary for retaining good antigen binding. Thus, the general humanizing methods described by Winter I have the disadvantage of frequently leading to inactive antibodies because these references do not provide information needed to rationally select among the many possible human framework sequences, those most likely to support antigen binding required by a particular CDR region from a non-human antibody.

U.S. Pat. No. 5,693,761 (Queen) discloses one refinement on Winter I for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable domain sequences are compared to a particular mouse sequence and the percentage identity between correspondent framework residues is calculated. The human variable domain with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6 Å of any CDR residue.

Another example approach for identifying criticality of amino acids in framework sequences is disclosed by U.S. Pat. No. 5,821,337 and by U.S. Pat. No. 5,859,205. These references disclose specific Kabat residue positions in the framework, which, in a humanized antibody may require substitution with the corresponding mouse amino acid to preserve avidity. One of the disadvantages of the refinements by Queen, and others is that a very large number of human framework sequences are required for comparison, and/or the guidelines for preserving critical amino acid residues are not completely sufficient to predict functionality. Accordingly, the resulting frameworks constructed, which are part human and part mouse, still frequently exhibit human immunogenicity or lowered antigen binding, thereby requiring numerous iterations in framework construction to obtain a suitable framework for therapeutic uses.

Humanized antibodies are typically prepared by replacing regions of mouse antibodies that are unimportant for antigen specificity with a human counterpart. The resulting humanized antibodies have residual murine sequences which, when administered to a human patient, often elicit immunological responses in the patient (human anti-mouse response). Therefore, it is desirable to prepare fully human antibodies that are void of non-human sequences. Fully human antibodies have been reported, obtained by means such as: construction and screening of a human antibody library using the phage display technique; by grafting lymphocytes from immunized human donors into severe combined immunodeficient (SCID) mice; or by engineering transgenic mice harboring human immunoglobulin genes (van Dijk et al., 2001). However, the diversity of such fully human libraries that has been achieved is at most about 10¹⁰ members. Therefore, there is a strong need in the art for fully human antibody display libraries that have achieved much higher diversity.

Fully human antibodies against pathogens have also been isolated by extensive screening of cord blood, which contains a natural poly-reactive IgM repertoire (U.S. Pat. No. 6,391,635). These methods, however, either produce antibodies with low affinities or depend on human donors with a desired immune response.

A variety of recombinant techniques for antibody preparation which do not rely on injection of an antigen into an animal have been developed. For example, it is possible to generate and select libraries of recombinant antibodies in phage or similar vectors. (Winter et al. “Making Antibodies by Phage Display Technology” Ann. Rev. Immunol. 12:433-55, 1994). Bacteriophage antibody libraries have also been produced for making high affinity human antibodies by chain shuffling (Marks et al., Biotechniques 10:779-782, 1992).

In general, the libraries include repertoires of V genes (e.g., harvested from populations of lymphocytes or assembled in vitro) which are cloned for display of associated heavy and light chain variable domains on the surface of filamentous bacteriophage. Phages are selected by binding to an antigen. Soluble antibodies are expressed from phage infected bacteria and the antibody can be improved, such as, by mutagenesis.

Although methods of producing antibodies by making, screening and evolving antibodies and antibody libraries are established, it would be desirable to have fully formed human antibody libraries with higher diversity.

Antibodies typically comprise two large heavy chains and two small light chains. There are five types of mammalian Ig heavy chain. They are denoted by the Greek letters: α, δ, ε, γ, and μ. The type of heavy chain present defines the class of antibody—IgA, IgD, IgE, IgG, and IgM. Each heavy chain comprises a constant region and a variable region. There are two types of light chains—lambda (λ) and kappa (κ). Each light chain comprises a constant region and a variable region. The constant region is identical in all antibodies of the same isotype. The variable region differs in antibodies produced by different B cells, but is identical for all antibodies produced by a single B cell.

The portion of the antibody that binds to an antigen (the antigen binding site) is contained in the Fab (fragment, antigen binding) region. The Fab region is composed of (a) a heavy chain constant and variable domain, and (b) a light chain constant and variable domain. The variable domain is referred to as the FV region. The variable domain on the light chain is abbreviated as VL. The variable domain on the heavy chain is abbreviated as VH.

The portion of the antibody that modulates immune cell activity is called the Fc (Fragment, crystallizable) region. The Fc region is composed of the constant regions of two heavy chains

### SUMMARY

The present disclosure provides a method of isolating a functional part of an immunoglobulin that specifically binds to an antigen, comprising: (a) transforming a plurality of cells with a nucleic acid sequence encoding a functional part of an immunoglobulin; (b) isolating cells that were transformed with a nucleic acid sequence to generate a population of host cells; (c) contacting the population of host cells with an antigen; (d) selecting a host cell that specifically binds to the antigen; and (e) isolating the nucleic acid sequence that encodes the functional part of an immunoglobulin. In some embodiments, the functional part of an immunoglobulin is a light chain of an immunoglobulin, a heavy chain of an immunoglobulin, a Fab domain, an Fv domain, or a combination thereof. In some embodiments, the functional part of an immunoglobulin is the variable portion of a light chain of an immunoglobulin, the constant portion of a light chain of an immunoglobulin, the variable portion of a heavy chain of an immunoglobulin, the constant portion of a heavy chain of an immunoglobulin or a combination thereof. In some embodiments, the nucleic acid sequence encoding the functional part of an immunoglobulin further comprises a transmembrane domain sequence. In some embodiments, the nucleic acid sequence encoding the functional part of an immunoglobulin is contained within a plasmid. In some embodiments, the plasmid further comprises a mammalian episomal origin of replication, a promoter, an antibiotic resistance gene, or a combination thereof. In some embodiments, the host cell is a bacterial cell, a yeast cell, or mammalian cell. In some embodiments, the antigen is labeled with a fluorescent molecule, a linker molecule or a magnetic particle. In some embodiments, selecting the host cell that interacts with the antigen is performed by magnetic separation or flow cell sorting.

Disclosed herein, in certain embodiments, is a method of isolating an immunoglobulin that specifically binds to an antigen, comprising: (a) transforming a plurality of cells with a nucleic acid sequence selected from: (i) a nucleic acid sequence encoding a heavy chain of an immunoglobulin, (ii) a nucleic acid sequence encoding a light chain of an immunoglobulin, or (iii) a nucleic acid sequence encoding a heavy chain of an immunoglobulin and a nucleic acid sequence encoding a light chain of an immunoglobulin; (b) isolating cells that were transformed with a nucleic acid sequence to generate a population of host cells; (c) contacting the population of host cells with an antigen; (d) selecting a host cell that specifically binds to the antigen; (e) isolating the nucleic acid sequence. In some embodiments, the nucleic acid sequence encoding the heavy chain, the light chain, or both further comprises a transmembrane domain sequence. In some embodiments, the nucleic acid sequence encoding the heavy chain and the nucleic acid sequence encoding the light chain are contained within a plasmid. In some embodiments, the nucleic acid sequence encoding the heavy chain is contained within a first plasmid, and the nucleic acid sequence encoding the light chain is contained within a second plasmid. In some embodiments, the plasmid further comprises a mammalian episomal origin of replication, a promoter, an antibiotic resistance gene, or a combination thereof. In some embodiments, the host cell is a bacterial, yeast or mammalian cell. In some embodiments, the antigen is labeled with a fluorescent molecule, a linker molecule or a magnetic particle. In some embodiments, selecting the host cell that interacts with the antigen is performed by magnetic separation or flow cell sorting.

### BRIEF DESCRIPTION OF THE DRAWINGS

FIG. 1 illustrates the pIgH vector.

FIG. 2 illustrates the pIgL vector.

FIG. 3 illustrates the pIgH&L vector.

FIG. 4 illustrates the pscFv vector.

FIG. 5 illustrates the pFab vector.

FIG. 6 illustrates the pIgHFab vector.

### DETAILED DESCRIPTION

Disclosed herein, in certain embodiments, is a method of isolating a functional part of an immunoglobulin, comprising: (a) transforming a cell with a nucleic acid sequence encoding a functional part of an immunoglobulin to generate a host cell; (b) contacting the host cell with an antigen; (c) selecting a host cell that interacts with the antigen; and (d) isolating the nucleic acid sequence that encodes the functional part of an immunoglobulin. In some embodiments, the functional part of an immunoglobulin is a light chain of an immunoglobulin, a heavy chain of an immunoglobulin, or a combination thereof. In some embodiments, the functional part of an immunoglobulin is the variable portion of a light chain of an immunoglobulin, the constant portion of a light chain of an immunoglobulin, the variable portion of a heavy chain of an immunoglobulin, the constant portion of a heavy chain of an immunoglobulin or a combination thereof.

Disclosed herein, in certain embodiments, is a method of isolating an antibody, comprising: (a) transforming a cell with (i) a nucleic acid sequence encoding a heavy chain of an immunoglobulin, and (ii) a nucleic acid sequence encoding a light chain of an immunoglobulin, to generate a host cell; (b) contacting the host cell with an antigen; (c) selecting a host cell that interacts with the antigen; (d) isolating the nucleic acid sequences that encode the heavy chain and the light chain.

Further disclosed herein, in certain embodiments, are novel vector designs, constructs and approaches to construct antibody libraries that display antibodies, such as full-length immunoglobulins, single chain antibody (SCA), scFv, or Fab on the host cell surface.

### Definitions

The phrase “specifically binds” when referring to the interaction between a binding molecule (i.e., the agent; e.g., a peptide or peptide mimetic) and a protein or polypeptide or epitope, typically refers to a binding molecule that recognizes and detectably specifically binds with high affinity to the target of interest. Preferably, under designated or physiological conditions, the specified antibodies or binding molecules bind to a particular polypeptide, protein or epitope yet does not bind in a significant or undesirable amount to other molecules present in a sample. In other words the specified antibody or binding molecule does not undesirably cross-react with non-target antigens and/or epitopes. A variety of immunoassay formats are used to select antibodies or other binding molecule that are immunoreactive with a particular polypeptide and have a desired specificity. For example, solid-phase ELISA immunoassays, BIAcore, flow cytometry and radioimmunoassays are used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that are used to determine or assess immunoreactivity and specificity.

“Selective binding,” “selectivity,” and the like refer the preference of agent to interact with one molecule as compared to another. Preferably, interactions between an agent disclosed herein and proteins are both specific and selective. Note that in some embodiments an agent is designed to “specifically bind” and “selectively bind” two distinct, yet similar targets without binding to other undesirable targets.

The terms “polypeptide,” “peptide” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds.

The terms “motif” and “domain” are used interchangeably. As used herein, they mean a discrete, contiguous or non-contiguous portion of a polypeptide that folds independently of the rest of the polypeptide and possesses its own function.

The term “disruption” means to interfere with the function of. For example, to disrupt a motif/domain means to interfere with the function of the motif/domain.

The term “antigen” refers to a substance that is capable of inducing the production of an antibody. In some embodiments an antigen is a substance that specifically binds to an antibody variable region.

The terms “antibody” and “antibodies” refer to monoclonal antibodies, polyclonal antibodies, bi-specific antibodies, multispecific antibodies, grafted antibodies, human antibodies, humanized antibodies, synthetic antibodies, chimeric antibodies, camelized antibodies, single-chain Fvs (scFv), single chain antibodies, Fab fragments, F(ab′) fragments, disulfide-linked Fvs (sdFv), intrabodies, and anti-idiotypic (anti-Id) antibodies and antigen-binding fragments of any of the above. In particular, antibodies include immunoglobulin molecules and immunologically active fragments of immunoglobulin molecules, i.e., molecules that contain an antigen binding site. Depending on the amino acid sequence of the constant motif/domain of their heavy chains, immunoglobulins can be assigned to different classes. The heavy-chain constant motif/domains (Fc) that correspond to the different classes of immunoglobulins are called α, δ, ε, γ, and μ, respectively. The subunit structures and three-dimensional configurations of different classes of immunoglobulins are well known. Immunoglobulin molecules are of any type (e.g., IgG, IgE, IgM, IgD, IgA and IgY), class (e.g., IgG₁, IgG₂, IgG₃, IgG₄, IgA₁ and IgA₂) or subclass. The terms “antibody” and “immunoglobulin” are used interchangeably in the broadest sense. In some embodiments an antibody is part of a larger molecule, formed by covalent or non-covalent association of the antibody with one or more other proteins or peptides.

### Vectors, Cells and Libraries

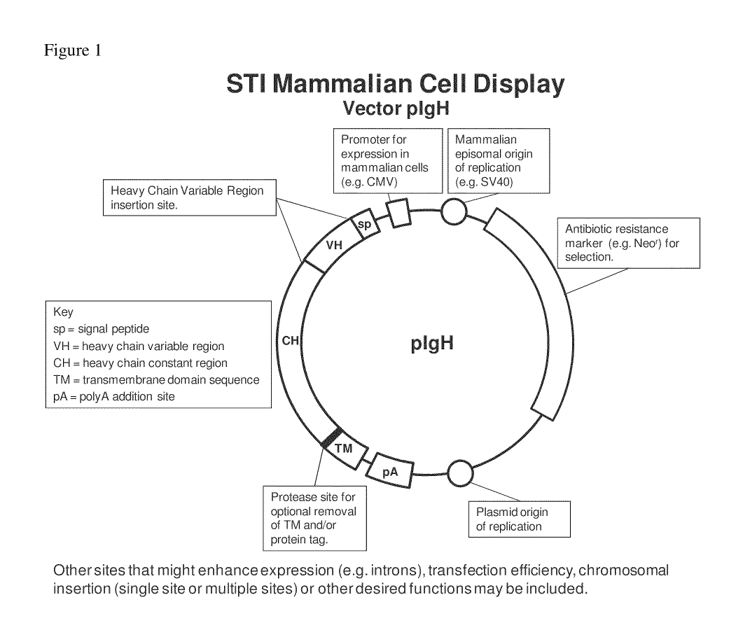

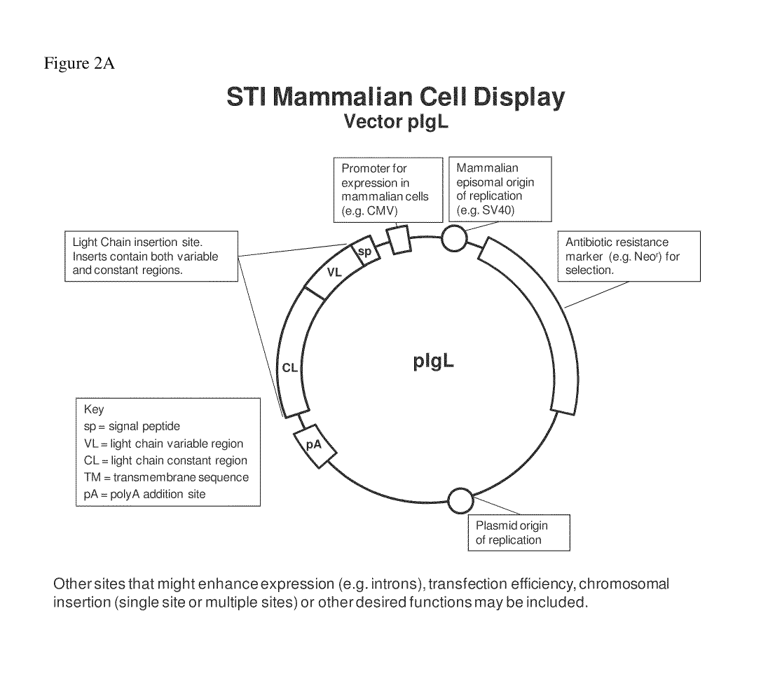

In one embodiment, the vector named as pIgH (FIG. 1), which comprises a mammalian episomal origin of replication (such as SV40 ori), an antibiotic resistance marker for antibiotic selection (such as neomycin gene NeoR), and a plasmid origin of replication. The pIgH comprises also a promoter for driven gene expression in mammalian cells (such as CMV promoter), which drives the down-stream full-length immunoglobulin heavy chain gene expression. The pIgH comprises a constant region (CH) sequence of the heavy chain, which at its c-termini a trans-membrane (TM) sequence (such as PDGFR beta trans-membrane domain) for anchoring the expressed immunoglobulin heavy chain onto the mammalian host cell surface built into the expression vector. The variable domain sequence of the heavy chain of the immunoglobulin gene (VH) or VH gene library inserts can be recombinantly inserted into the insertion sites as designed in the pIgH vector. The said vector comprises optionally an enzymatic digestion site (such as thrombin site) for protease cleavage to release the anchored antibody into the spent media.

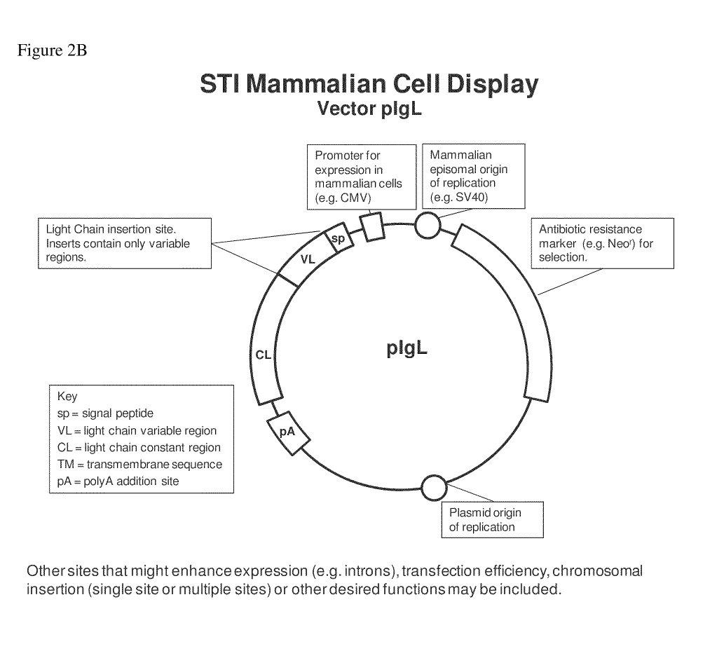

In another embodiment, the vector named as pIgL (FIG. 2), which comprises a mammalian episomal origin of replication (such as SV40 ori), an antibiotic resistance marker for antibiotic selection (such as neomycin gene NeoR), and a plasmid origin of replication. The pIgL comprises also a promoter for driven gene expression in mammalian cells (such as CMV promoter), which drives the down-stream full-length immunoglobulin light chain gene expression. The pIgL comprises a variable domain and a constant region (CL) sequence of the light chain.

In one embodiment, a library of variable domains of the heavy chains (VH Library) is inserted into pIgH vector of FIG. 1 to generate a FL immunoglobulin heavy chain library (IgH Library).

In another embodiment, a single variable domain sequence of a selected heavy chain (sVH) is inserted into the pIgH vector of FIG. 1 to generate a single FL immunoglobulin heavy chain (sIgH).

In one embodiment, a library of FL light chains is inserted into pIgL vector of FIG. 2 to generate a FL immunoglobulin light chain library (IgL Library).

In another embodiment, a single, selected FL light chain is inserted into the pIgH vector of FIG. 2 to generate a single FL immunoglobulin light chain (sIgL).

In a preferred embodiment, a FL IgH Library and a FL IgL library are co-transfected into a mammalian cell culture and FL IgH and FL IgL genes are co-expressed in individual co-transfected mammalian cells and displayed onto the cell surface. Each individual cell may express hundreds (10²) to hundreds thousands (10⁵) fully assembled FL immunoglobulins anchored on the cell surface by the TM domain. A cell culture of 10⁶ to 10¹⁰ cells potentially expresses and displays 10⁸ to 10¹⁵ fully assembled FL immunoglobulins on cell surface.

In another preferred embodiment, a FL IgH Library and a FL sIgL are co-transfected into a mammalian cell culture and FL IgH and FL sIgL genes are co-expressed in individual co-transfected mammalian cells and displayed onto the cell surface. Each individual cell may express hundreds to hundreds thousands fully assembled FL immunoglobulins anchored on the cell surface by the TM domain. All of the FL immunoglobulins in each of the mammalian cells may comprise a single common FL light chain (sIgL) and different FL IgH chain on the surface anchored fully assembled immunoglobulins. Each individual cell may express hundreds (10²) to hundreds thousands (10⁵) fully assembled FL immunoglobulins, comprising a single common FL light chain of sIgL and different FL IgH, anchored on the cell surface by the TM domain. A cell culture of 10⁶ to 10¹⁰ cells potentially expresses and displays 10⁸ to 10¹⁵ fully assembled FL immunoglobulins, all comprising a sIgL, on cell surface.

In yet another preferred embodiment, a FL sIgH and a FL IgL Library are co-transfected into a mammalian cell culture and FL sIgH and FL IgL genes are co-expressed in individual co-transfected mammalian cells and displayed onto the cell surface. Each individual cell may express hundreds to hundreds thousands fully assembled FL immunoglobulins anchored on the cell surface by the TM domain. All of the FL immunoglobulins in each of the mammalian cells may comprise a single common FL heavy chain (sIgH) and different FL IgL chain on the surface anchored fully assembled immunoglobulins. Each individual cell may express hundreds (10²) to hundreds thousands (10⁵) fully assembled FL immunoglobulins, comprising a single common FL heavy chain of sIgH and different FL IgL, anchored on the cell surface by the TM domain. A cell culture of 10⁶ to 10¹⁰ cells potentially expresses and displays 10⁸ to 10¹⁵ fully assembled FL immunoglobulins, all comprising a sIgH, on cell surface.

In one preferred embodiment, a single FL heavy chain immunoglobulin (sIgH) or a library of FL heavy chain immunoglobulin (IgH Library) are transfected into mammalian cells. The transfected mammalian cells are made permanent by antibiotics selection (such as G418 drug selection when neomycin resistance gene is expressed. The permanent mammalian cells expressing one or a plural of IgH are termed hereof as IgH-Expressing Line.

In another preferred embodiment, a single FL light chain immunoglobulin (sIgL) or a library of FL light chain immunoglobulin (IgL Library) are transfected into mammalian cells. The transfected mammalian cells are made permanent by antibiotics selection (such as G418 drug selection when neomycin resistance gene is expressed. The permanent mammalian cells expressing one or a plural of IgL are termed hereof as IgL-Expressing Line.

In one preferred embodiment, an IgH Library are transfected into an IgL-Expressing Line so that the transfect cells of IgL-Expressing Line express fully assembled immunoglobulins in the transfected IgL-Expressing Line cells, with each comprising hundreds (10²) to hundreds thousands (10⁵) IgHs and a single IgL. A cell culture of 10⁶ to 10¹⁰ cells may produce 10e8 to 10¹⁵ different fully assembled FL immunoglobulins on the cell surface. These transfected cells are subjected to further antigen bio-panning and binding selection.

In another preferred embodiment, an IgL Library are transfected into an IgH-Expressing Line so that the transfect cells of IgH-Expressing Line express fully assembled immunoglobulins in the transfected IgH-Expressing Line cells, with each comprising hundreds (10²) to hundreds thousands (10⁵) IgLs and a single IgH. A cell culture of 10⁶ to 10¹⁰ cells may produce 10⁸ to 10¹⁵ different fully assembled FL immunoglobulins on the cell surface. These transfected cells are subjected to further antigen bio-panning and binding selection.

In one embodiment, the pIgH and pIgL vectors, of different backbones (such as they contain different antibiotics resistant genes), are used to construct both full-length heavy and light chain immunoglobulin libraries. The heavy and light chain immunoglobulin libraries are constructed initially by transforming the pIgH and pIgL constructs in prokaryotic cells and the isolated vectors comprising either heavy or light chain immunoglobulin genes in plasmids form. The pIgH and pIgL are co-transfected into a mammalian cell for co-expression of multiple different types of heavy chains and light chains in each individual mammalian cell. The cells expressing a plural of properly configured and assembled immunoglobulins are screened and selected for proper binding against selected target antigens. The selected subpopulation of the vectors expressing the immunoglobulins is recovered from the cell episomally or cytoplasmically for further analysis and selection process. In the event the pIgH and pIgL use two different antibiotics drug resistance selection marker genes, the pIgH and pIgL plasmids can be easily separated by different antibiotics selection.

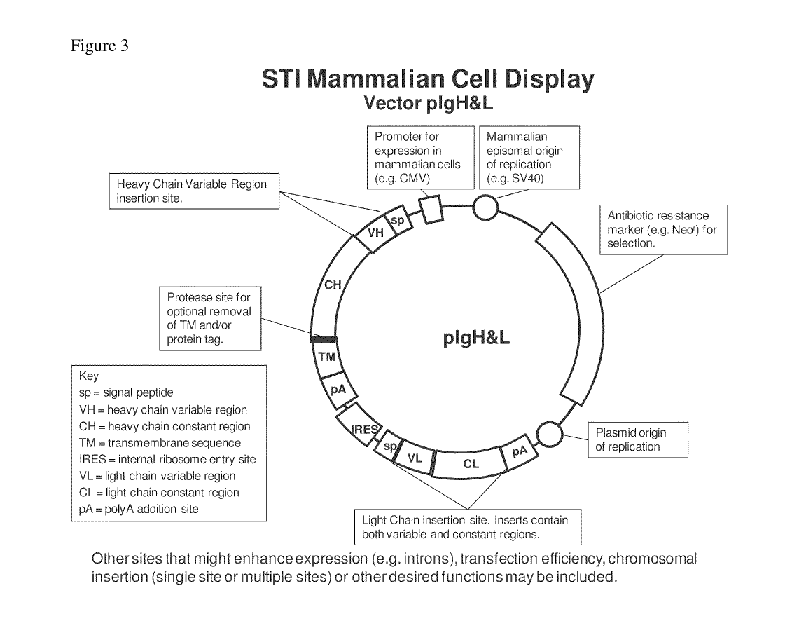

In one embodiment, the vector named as pIgH&L (FIG. 3), which comprises a mammalian episomal origin of replication (such as SV40 ori), an antibiotic resistance marker for antibiotic selection (such as neomycin gene NeoR), and a plasmid origin of replication. The pIgH&L comprises also a promoter for driven gene expression in mammalian cells (such as CMV promoter), which drives the down-stream full-length immunoglobulin heavy and light chain gene co-expression. The heavy and light chain co-expression is achievable by an internal ribosomal entry site (IRES) linked in between the FL H and L chains. The FL H chain comprises a TM sequence at its c-termini for anchoring the FL immunoglobulin H chain onto the mammalian cell surface. The said vector comprises optionally an enzymatic digestion site (such as thrombin site) for protease cleavage to release the anchored antibody into the spent media.

In a preferred embodiment, a library of variable domain sequences of the heavy chain are inserted into the VH insertion site of pIgH&L to form an IgH Library, while a single common FL light chain is inserted in the vector pIgH&L, which upon transfection into a mammalian cell culture co-expresses a library of FL IgH and a single common FL sIgL genes in individual transfected mammalian cells and displayed onto the cell surface. Each individual cell may express hundreds (10²) to hundreds thousands (10⁵) fully assembled FL immunoglobulins anchored on the cell surface by the TM domain. All of the FL immunoglobulins in each of the mammalian cells may comprise a single common FL light chain (sIgL) and different FL IgH chain on the surface anchored fully assembled immunoglobulins. A cell culture of 10⁶ to 10¹⁰ cells potentially expresses and displays 10⁸ to 10¹⁵ fully assembled FL immunoglobulins, all comprising a sIgL, on cell surface.

In another preferred embodiment, a library of FL light chain are inserted into the light chain insertion site of pIgH&L to form an IgL Library, while a single common FL heavy chain is inserted in the vector pIgH&L, which upon transfection into a mammalian cell culture co-expresses a library of FL IgL and a single common FL sIgH genes in individual transfected mammalian cells and displayed onto the cell surface. Each individual cell may express hundreds (10²) to hundreds thousands (10⁵) fully assembled FL immunoglobulins anchored on the cell surface by the TM domain. All of the FL immunoglobulins in each of the mammalian cells may comprise a single common FL heavy chain (sIgH) and different FL IgL chain on the surface anchored fully assembled immunoglobulins. A cell culture of 10⁶ to 10¹⁰ cells potentially expresses and displays 10⁸ to 10¹⁵ fully assembled FL immunoglobulins, all comprising a sIgH, on cell surface.

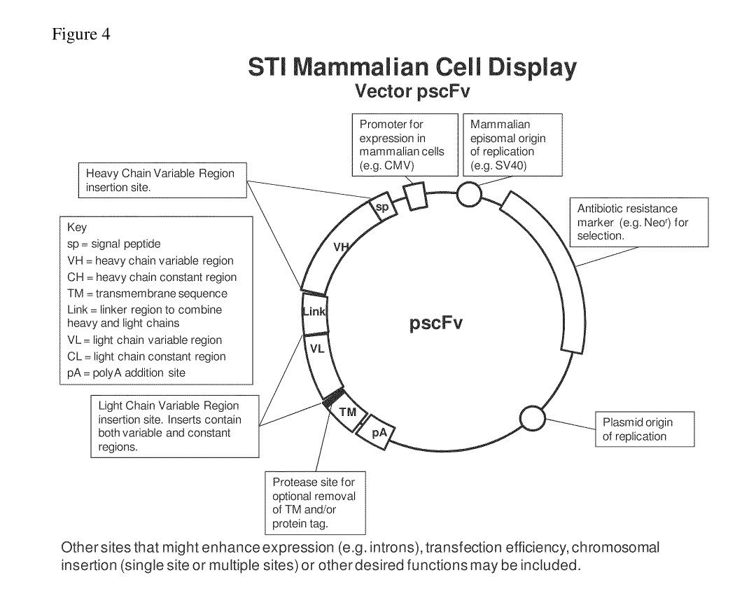

In one embodiment, the vector named as pscFv (FIG. 4), which comprises a mammalian episomal origin of replication (such as SV40 ori), an antibiotic resistance marker for antibiotic selection (such as neomycin gene NeoR), and a plasmid origin of replication. The pscFv comprises also a promoter for driven gene expression in mammalian cells (such as CMV promoter), which drives the down-stream single chain antibody (SCA) in the form of scFv, which is formed by linking variable heavy chain domain (VH) with variable light chain domain (VL) with a peptide linker. The scFv sequence comprises a TM sequence at its c-termini for anchoring the scFv onto the mammalian cell surface. The said vector comprises optionally an enzymatic digestion site (such as thrombin site) or tagging peptide (such a c-myc tag) for protease cleavage to release the anchored antibody into the spent media or tag peptide detection. In one embodiment, the VH inserts are of a library of VH domain sequences (VH Library) and the VL insert is a single VL sequence (sVL), the transfection of library comprising a VH Library and a sVL into a cell culture to express the scFv library and display on the cell surface via TM anchoring. A library of VH Library containing hundreds (10²) to hundreds millions (10⁸) of VH inserts and a common sVL are transfected into a cell culture with each individual cell expresses and displays hundreds (10²) to hundreds thousands (10⁵) different scFv comprising a different VH and a common sVL. Vice versa, the VL inserts are of a library of VL domain sequences (VL Library) and the VH insert is a single VH sequence (sVH), the transfection of library comprising a VL Library and a sVH into a cell culture to express the scFv library and display on the cell surface via TM anchoring. A library of VL Library containing hundreds (10²) to hundreds millions (10⁸) of VL inserts and a common sVH are transfected into a cell culture with each individual cell expresses and displays hundreds (10²) to hundreds thousands (10⁵) different scFv comprising a different VL and a common sVH.

In another embodiment, the pscFv vector is a phage display vector, which comprises an antibiotic resistance marker for antibiotic selection (such as Ampicillin gene AmpR), a plasmid origin of replication. The pscFv comprises also a promoter for driven gene expression in E. coli cells, which drives the down-stream scFv gene expression. The scFv of VH-Peptide Linker-VL is operatively linked with a phage coat protein such as pIII, pVII, pVIII, or pIX so the display of scFv is achieved via linked coat protein onto the phage particle surface. A library of VH Library containing hundreds (10²) to hundreds millions (10⁸) of VH inserts and a common sVL are transformed into an E. coli cell culture with each individual E. coli cell expresses and displays one single scFv comprising a different VH and a common sVL. Vice versa, the VL inserts are of a library of VL domain sequences (VL Library) and the VH insert is a single VH sequence (sVH), the transformation of library comprising a VL Library and a sVH into an E. coli cell culture to express the scFv library and display on the phage particle via fused phage coat protein. A library of VL Library containing hundreds (10²) to hundreds millions (10⁸) of VL inserts and a common sVH are transformed into an E. coli cell culture with each individual E. coli cell expresses and displays one single scFv comprising a different VL and a common sVH.

In yet another embodiment, the pscFv vector is a yeast display vector, which comprises an antibiotic resistance marker for antibiotic selection (such as Ampicillin gene AmpR), a plasmid origin of replication. The pscFv comprises also a promoter for driven gene expression in E. coli cells, which drives the down-stream scFv gene expression. The scFv of VH-Peptide Linker-VL is operatively linked with a yeast surface protein so the display of scFv is achieved via linked yeast surface protein onto the yeast cell surface. A library of VH Library containing hundreds (10²) to hundreds millions (10⁸) of VH inserts and a common sVL are transformed into an yeast cell culture with each individual yeast cell expresses and displays one single scFv comprising a different VH and a common sVL. Vice versa, the VL inserts are of a library of VL domain sequences (VL Library) and the VH insert is a single VH sequence (sVH), the transformation of library comprising a VL Library and a sVH into a yeast cell culture to express the scFv library and display on the yeast surface via fused yeast surface protein. A library of VL Library containing hundreds (10²) to hundreds millions (10⁸) of VL inserts and a common sVH are transformed into an yeast cell culture with each individual yeast cell expresses and displays one single scFv comprising a different VL and a common sVH.

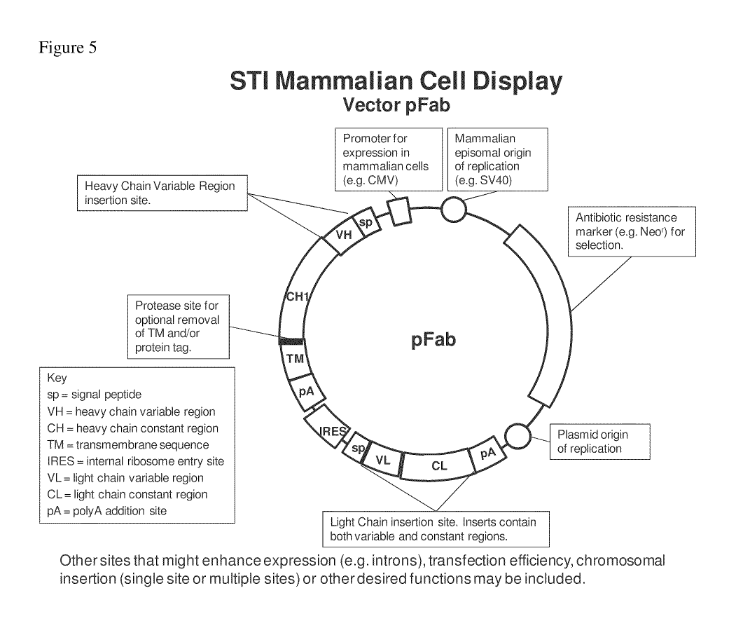

In one embodiment, the vector named as pFab (FIG. 5), which comprises a mammalian episomal origin of replication (such as SV40 ori), an antibiotic resistance marker for antibiotic selection (such as neomycin gene NeoR), and a plasmid origin of replication. The pFab comprises also a promoter for driven gene expression in mammalian cells (such as CMV promoter), which drives the down-stream Fab gene expression. The Fab, VH-CH1 and FL light chain of VL-CL, co-expression is achievable by an internal ribosomal entry site (IRES) linked in between the VH-CH1 and VL-CL. Either the VH-CH1 chain or VL-CL light chain comprises a TM sequence at its corresponding c-termini for anchoring the Fab onto the mammalian cell surface. The said pFab vector comprises optionally an enzymatic digestion site (such as thrombin site) for protease cleavage to release the anchored antibody into the spent media or protein tag site for detection of the protein tag.

In a preferred embodiment, a library of VH-CH1 inserts and a library of VL-CL inserts are inserted into the pFab vector and transfected into a mammalian cell culture. The VH-CH1 and VL-CL genes are co-expressed in individual co-transfected mammalian cells and the Fab are assembled and displayed onto the cell surface. Each individual cell may express hundreds (10²) to hundreds thousands (10⁵) fully assembled Fab anchored on the cell surface by the TM domain. A cell culture of 10⁶ to 10¹⁰ cells potentially expresses and displays 10⁸ to 10¹⁵ fully assembled Fab on cell surface.

In another preferred embodiment, a library of VH-CH1 inserts and a single VL-CL insert are inserted into the pFab vector and transfected into a mammalian cell culture. The VH-CH1 genes and single VL-CL gene are co-expressed in individual co-transfected mammalian cells and the Fab are assembled and displayed onto the cell surface. Each individual cell may express hundreds (10²) to hundreds thousands (10⁵) fully assembled Fab, each comprising a different VH-CH1 and a common VL-CL insert, anchored on the cell surface by the TM domain. A cell culture of 10⁶ to 10¹⁰ cells potentially expresses and displays on cell surface 10⁸ to 10¹⁵ fully assembled Fab with each comprising a different VH-CH1 and a common VL-CL.

In yet another preferred embodiment, a single of VH-CH1 insert and a library of VL-CL inserts are inserted into the pFab vector and transfected into a mammalian cell culture. The single common VH-CH1 gene and VL-CL genes are co-expressed in individual co-transfected mammalian cells and the Fab are assembled and displayed onto the cell surface. Each individual cell may express hundreds (10²) to hundreds thousands (10⁵) fully assembled Fab, each comprising a single common VH-CH1 fragment and a different VL-CL fragment, anchored on the cell surface by the TM domain. A cell culture of 10⁶ to 10¹⁰ cells potentially expresses and displays on cell surface 10⁸ to 10¹⁵ fully assembled Fab with each comprising a common VH-CH1 and a different VL-CL fragments.

In another embodiment, the pFab vector of FIG. 5 is a phage display vector, which comprises an antibiotic resistance marker for antibiotic selection (such as Ampicillin gene AmpR), a plasmid origin of replication. The pscFv comprises also a promoter for driven gene expression in E. coli cells, which drives the down-stream Fab gene expression. The Fab, VH-CH1 and FL light chain of VL-CL, co-expression is achievable by an internal ribosomal entry site (IRES) linked in between the VH-CH1 and VL-CL. The Fab of VH-CH1 and VL-CL is operatively linked with a phage coat protein such as pIII, pVII, pVIII, or pIX so the display of Fab is achieved via linked coat protein onto the phage particle surface. A library of VH-CH1 Library containing hundreds (10²) to hundreds millions (10⁸) of VH-CH1 inserts and a single common FL light chain sVL-CL are transformed into an E. coli cell culture with each individual E. coli cell expresses and displays one single Fab comprising a different VH-CH1 and a common sVL-CL. Vice versa, the FL VL-CL inserts are of a library of FL VL-CL light chain sequences (VL-CL Library) and the VH insert is a single VH-CH1 sequence (sVH-CH1), the transformation of library comprising a VL-CL Library and a sVH-CH1 into an E. coli cell culture to express the Fab library and display on the phage particle via fused phage coat protein. A library of VL-CL Library containing hundreds (10²) to hundreds millions (10⁸) of VL-CL inserts and a common sVH-CH1 are transformed into an E. coli cell culture with each individual E. coli cell expresses and displays one single Fab comprising a different VL-CL and a common sVH-CH1.

In yet another embodiment, the pFab vector is a yeast display vector, which comprises an antibiotic resistance marker for antibiotic selection (such as neomycin gene AmpR), a plasmid origin of replication. The pFab comprises also a promoter for driven gene expression in yeast cells, which drives the down-stream Fab gene expression. The Fab of VH-CH1 and VL-CL is operatively linked with a yeast surface protein, with either VH-CH1 or VL-CL, so the display of Fab is achieved via linked yeast surface protein onto the yeast cell surface. A library of VH-CH1 Library containing hundreds (10²) to hundreds millions (10⁸) of VH-CH1 inserts and a common sVL-CL are transformed into an yeast cell culture with each individual yeast cell expresses and displays one single Fab comprising a different VH-CH1 and a common sVL-CL. Vice versa, the VL-CL inserts are of a library of FL VL-CL light chain sequences (VL-CL Library) and the VH insert is a single VH-CH1 sequence (sVH-CH1), the transformation of library comprising a VL-CL Library and a sVH-CH1 into an yeast cell culture to express the Fab library and display on the yeast surface via fused yeast surface protein. A library of VL Library containing hundreds (10²) to hundreds millions (10⁸) of VL-CL inserts and a common sVH-CH1 are transformed into an yeast cell culture with each individual yeast cell expresses and displays one single Fab comprising a different VL-CL and a common sVH-CH1.

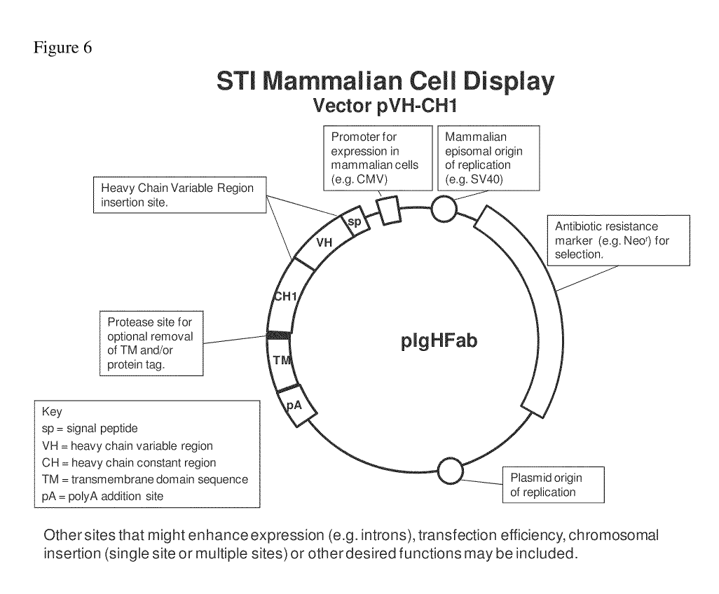

In one embodiment, the vector named as pVH-CH1 (FIG. 6), which comprises a mammalian episomal origin of replication (such as SV40 ori), an antibiotic resistance marker for antibiotic selection (such as neomycin gene NeoR), and a plasmid origin of replication. The pVH-CH1 comprises also a promoter for driven gene expression in mammalian cells (such as CMV promoter), which drives the down-stream VH-CH1 gene expression. The VH-CH1 may optionally comprise a TM sequence at its c-termini for anchoring the VH-CH1 onto the mammalian cell surface. The said pVH-CH1 vector comprises optionally an enzymatic digestion site (such as thrombin site) for protease cleavage to release the anchored antibody into the spent media or protein tag site for detection of the protein tag.

In another embodiment, the vector named as pIgL of FIG. 2, comprises a VL-CL insert that may optionally comprise a TM sequence at its c-termini for anchoring the FL VL-CL onto the mammalian cell surface. The said pVL-CL vector comprises optionally an enzymatic digestion site (such as thrombin site) for protease cleavage to release the anchored antibody into the spent media or protein tag site for detection of the protein tag.

In one embodiment, a library of VH-CH1 inserts is inserted into pVH-CH1 vector of FIG. 6 to generate a pVH-CH1 library (pVH-CH1 Library).

In another embodiment, a single VH-CH1 (sVH-CH1) insert is inserted into the pVH-CH1 vector of FIG. 6 to generate a single common pVH-CH1 (sVH-CH1).

In a preferred embodiment, a pVH-CH1 Library and a FL IgL library are co-transfected into a mammalian cell culture and VH-CH1 and FL IgL genes are co-expressed in individual co-transfected mammalian cells and displayed onto the cell surface. Each individual cell may express hundreds (10²) to hundreds thousands (10⁵) fully assembled Fabs anchored on the cell surface by the TM domain. A cell culture of 10⁶ to 10¹⁰ cells potentially expresses and displays 10⁸ to 10¹⁵ fully assembled Fabs on cell surface.

In another preferred embodiment, a pVH-CH1 Library and a FL sIgL are co-transfected into a mammalian cell culture and VH-CH1 and FL sIgL genes are co-expressed in individual co-transfected mammalian cells and displayed onto the cell surface. Each individual cell may express hundreds to hundreds thousands fully assembled Fabs anchored on the cell surface by the TM domain. All of the Fabs in each of the mammalian cells may comprise a single common FL light chain (sIgL) and different VH-CH1 on the surface anchored fully assembled Fabs. Each individual cell may express hundreds (10²) to hundreds thousands (10⁵) fully assembled Fabs, comprising a single common FL light chain of sIgL and different VH-CH1 heavy chain fragment, anchored on the cell surface by the TM domain. A cell culture of 10⁶ to 10¹⁰ cells potentially expresses and displays 10⁸ to 10¹⁵ fully assembled Fabs, all comprising a sIgL, on cell surface.

In yet another preferred embodiment, a single common sVH-CH1 and a FL IgL Library are co-transfected into a mammalian cell culture and sVH-CH1fragment and FL IgL genes are co-expressed in individual co-transfected mammalian cells and displayed onto the cell surface. Each individual cell may express hundreds to hundreds thousands fully assembled Fabs anchored on the cell surface by the TM domain. All of the Fabs in each of the mammalian cells may comprise a single common VH-CH1 (sVH-CH1) and different FL IgL chain on the surface anchored fully assembled Fabs. Each individual cell may express hundreds (10²) to hundreds thousands (10⁵) fully assembled Fabs, comprising a single common VH-CH1 and different FL IgL, anchored on the cell surface by the TM domain. A cell culture of 10⁶ to 10¹⁰ cells potentially expresses and displays 10⁸ to 10¹⁵ fully assembled Fabs, all comprising a sVH-CH1, on cell surface.

In one preferred embodiment, a single sVH-CH1 or a library of pVH-CH1 (pVH-CH1 Library) are transfected into mammalian cells. The transfected mammalian cells are made permanent by antibiotics selection (such as G418 drug selection when neomycin resistance gene is expressed. The permanent mammalian cells expressing one or a plural of VH-CH1 are termed hereof as VH-CH1-Expressing Line.

In one preferred embodiment, an pVH-CH1 Library are transfected into an IgL-Expressing Line so that the transfect cells of IgL-Expressing Line express fully assembled Fabs the transfected IgL-Expressing Line cells, with each comprising hundreds (10²) to hundreds thousands (10⁵) VH-CH1 inserts and a single IgL. A cell culture of 10⁶ to 10¹⁰ cells may produce 10⁸ to 10¹⁵ different fully assembled Fabs on the cell surface. These transfected cells are subjected to further antigen bio-panning and binding selection for the selected Fabs for a desired antigen.

In another preferred embodiment, an IgL Library are transfected into an VH-CH1-Expressing Line so that the transfect cells of VH-CH1-Expressing Line express fully assembled Fabs the transfected VH-CH1-Expressing Line cells, with each comprising hundreds (10²) to hundreds thousands (10⁵) IgLs and a single VH-CH1 fragment. A cell culture of 10⁶ to 10¹⁰ cells may produce 10⁸ to 10¹⁵ different fully assembled Fabs on the cell surface. These transfected cells are subjected to further antigen bio-panning and binding selection for the selected Fabs for a desired antigen.

In one embodiment, the pVH-CH1 and pIgL vectors, of different backbones (such as they contain different antibiotics resistant genes), are used to construct both VH-CH1 heavy chain fragment and FL light chain immunoglobulin libraries. The VH-CH1 and light chain immunoglobulin libraries are constructed initially by transforming the pVH-CH1 and pIgL constructs in prokaryotic cells and the isolated vectors comprising either heavy fragment or full-length light chain immunoglobulin genes in plasmids forms. The pVH-CH1 and pIgL are co-transfected into a mammalian cell for co-expression of multiple different types of VH-CH1 heavy chain fragments and FL light chains in each individual mammalian cell. The cells expressing a plural of properly configured and assembled Fabs are screened and selected for proper binding against selected target antigens. The selected subpopulation of the vectors expressing the Fabs is recovered from the cell episomally or cytoplasmically for further analysis and selection process. In the event the pVH-CH1 and pIgL use two different antibiotics drug resistance selection marker genes, the pVH-CH1 and pIgL plasmids can be easily separated by different antibiotics selection.

The bio-panning of the above mentioned different types of antibody libraries (FL IgG1, scFv or Fab libraries) against desired antigen can be carried out by selection of specific binding of the antibodies in the host cells with the desired antigen.

In one embodiment, the host cells are E. coli, wherein the antibody libraries are in a phage display vector(s). The E. coli cells that express binder antibodies (FL IgG1, scFv or Fab antibodies) are selected and the phage particles contained within the host E coli cells are recovered. The bio-panning process can be repeated to further enrich the phage particles that express the desired binder antibodies.

In another embodiment, the host cells are yeast cells, wherein the antibodies in the antibody libraries are displayed on the yeast cell surface. The desired antigen can be fluorescent labeled and incubated with the yeast cells contain the expressed antibody library. The yeast cells that bind with the fluorescent labeled antigen are selected by flow cell sorting (FACS). The cell sorting selection process can be repeated to further enrich the yeast cells containing the desired binder antibodies from the yeast antibody library.

In yet another embodiment, the host cells are mammalian cells, wherein the antibodies in the antibody libraries are displayed on the mammalian cell surface. The desired antigen can be fluorescent labeled and incubated with the mammalian cells contain the expressed antibody library. The mammalian cells that bind with the fluorescent labeled antigen are selected by flow cell sorting (FACS). The cell sorting selection process can be repeated to further enrich the mammalian cells containing the desired binder antibodies from the mammalian antibody library.

In one embodiment, the desired antigen is covalently linked with a magnetic particle. The magnetic particle linked with a desired antigen is incubated with the host cells, such as E. coli, yeast or mammalian cells that express an antibody library. The host cells contain binder antibodies are separated from the cells that contain non-binder antibodies and desired antibodies and their corresponding gene sequences are isolated. The bio-panning process employing the magnetic particles can be repeated to further enrich the host cells contain the desired binder antibodies.

We claim:

1. A method of isolating a functional part of an immunoglobulin that specifically binds to an antigen, comprising:
(a) transforming a cell with a nucleic acid sequence encoding a functional part of an immunoglobulin;
(b) isolating cells that were transformed with a nucleic acid sequence encoding a functional part of an immunoglobulin to generate a population of host cells;
(c) contacting the host cell with an antigen;
(d) selecting a host cell that specifically binds to the antigen; and
(e) isolating the nucleic acid sequence that encodes the functional part of an immunoglobulin.

2. The method of claim 1, wherein the functional part of an immunoglobulin is a light chain of an immunoglobulin, a heavy chain of an immunoglobulin, a Fab domain, a Fv domain, or a combination thereof.

3. The method of claim 1, wherein the functional part of an immunoglobulin is the variable portion of a light chain of an immunoglobulin, the constant portion of a light chain of an immunoglobulin, the variable portion of a heavy chain of an immunoglobulin, the constant portion of a heavy chain of an immunoglobulin or a combination thereof.

4. The method of claim 1, wherein the nucleic acid sequence encoding the functional part of an immunoglobulin further comprises a transmembrane domain sequence.

5. The method of claim 1, wherein the nucleic acid sequence encoding the functional part of an immunoglobulin is contained within a plasmid.

6. The method of claim 5, wherein the plasmid further comprises a mammalian episomal origin of replication, a promoter, an antibiotic resistance gene, or a combination thereof.

7. The method of claim 1, wherein the host cell is a bacterial cell, a yeast cell, or mammalian cell.

8. The method of claim 1, wherein the antigen is labeled with a fluorescent molecule, a linker molecule or a magnetic particle.

9. The method of claim 1, wherein selecting the host cell that interacts with the antigen is performed by magnetic separation or flow cell sorting.

10. A method of isolating an immunoglobulin that specifically binds to an antigen, comprising:
(a) transforming a plurality of cells with a nucleic acid sequence selected from:
i. a nucleic acid sequence encoding a heavy chain of an immunoglobulin,
ii. a nucleic acid sequence encoding a light chain of an immunoglobulin, or
iii. a nucleic acid sequence encoding a heavy chain of an immunoglobulin and a nucleic acid sequence encoding a light chain of an immunoglobulin;
(b) isolating cells that were transformed with a nucleic acid sequence to generate a population of host cells;
(c) contacting the population of host cells with an antigen;
(d) selecting a host cell that specifically binds to the antigen; and
(e) isolating the nucleic acid sequence.

11. The method of claim 10, wherein the immunoglobulin is a functional antibody.

12. The method of claim 10, wherein the immunoglobulin is a single chain antibody (SCA).

13. The method of claim 10, wherein the immunoglobulin is a scFv.

14. The method of claim 10, wherein the nucleic acid sequence encoding the heavy chain, the light chain, or both further comprises a transmembrane domain sequence.

15. The method of claim 10, wherein the nucleic acid sequence encoding the heavy chain and the nucleic acid sequence encoding the light chain are contained within a plasmid.

16. The method of claim 10, wherein the nucleic acid sequence encoding the heavy chain is contained within a first plasmid, and the nucleic acid sequence encoding the light chain is contained within a second plasmid.

17. The method of claim 12, wherein the plasmid further comprises a mammalian episomal origin of replication, a promoter, an antibiotic resistance gene, or a combination thereof.

18. The method of claim 10, wherein the host cell is a bacterial, yeast or mammalian cell.

19. The method of claim 10, wherein the antigen is labeled with a fluorescent molecule, a linker molecule or a magnetic particle.

20. The method of claim 10, wherein selecting the host cell that interacts with the antigen is performed by magnetic separation or flow cell sorting.
